(19) United States
(12) Patent Application Publication
(10) Pub. No.: US 2011/0243918 A1
(43) Pub. Date: Oct. 6, 2011

(54) TOPICAL MEDICATION FOR THE TREATMENT OF HEMORRHOIDS AND METHOD OF USE

(76) Inventor: Joseph P. Thornton, Las Vegas, NV (US)

(21) Appl. No.: 12/547,241

(22) Filed: Aug. 25, 2009

(51) Int. Cl. A61K 38/48

(52) U.S. Cl. 424/94.65

(57) ABSTRACT

A natural gel/ointment for the treatment of hemorrhoids. Bromelain is one primary active ingredient in the gel/ointment. A second active ingredient such as Hamamelis Virginiana (Witch Hazel) may be combined with the Bromelain. Secondary ingredients include alcohol, de-ionized water, Butylene Glycol, PEG-40 Hydrogenated Castor Oil, Hydroxyethylcellulose, Eucalyptus Globulus Oil, Aloe Barbadensis Leaf Extract, Chamomilla Rectutita Flower Extract, Camellia Sinensis Leaf Extract, Avena Sativa Kernel Flour, Benzophenone-4, Disodium EDTA, Sodium Citrate, Sodium Hydroxide, Citric Acid, Caprylyl Glycol, Chlorphenesin and Methylisothiazolinone. Based on case studies the gel/ointment outperforms other hemorrhoid medications.

### RELATED APPLICATIONS

This application is related to co-pending U.S. application Ser. No. 12/______ and U.S. application Ser. No. 12/______, both of which are filed herewith and incorporated herein in their entirety.

### FIELD OF THE INVENTION

The embodiments of the present invention relate to a natural, topical composition for the treatment of hemorrhoids.

### BACKGROUND

Hemorrhoids are swollen veins in the anal canal. Millions of people suffer from hemorrhoids and a majority of adults will suffer from hemorrhoids at one point in their life. Treatments for hemorrhoids range from surgery to topical ointments. Surgery includes rubber band ligation, cryosurgery and surgical hemorrhoidectomy. Non-surgical procedures include the application of heat, use of lasers or electric current to create scar tissue, or sclerotheraphy. Medicinal treatments include ointments and creams such as Preparation H®, medicated wipes and the like. Despite the various treatments, hemorrhoids continue to flourish.

Thus, there exists a need for natural treatment to more reliably and efficiently treating hemorrhoids.

### SUMMARY

Accordingly, one embodiment of the present invention is a natural, topical gel or ointment including at least one anti-inflammatory agent and in some embodiments two anti-inflammatory agents. In one embodiment, the gel/ointment includes one or both of Bromelain and Witch Hazel as the anti-inflammatory agents. Other ingredients are compounded with the Bromelain and Witch Hazel to form a gel/ointment composition as the delivery vehicle.

In one embodiment, the gel/ointment is applied up to three times daily until hemorrhoid symptoms dissipate. As evidenced by the case studies described below, the use of the natural gel/ointment reduces dramatically the swelling, bleeding, itching and other symptoms associated with hemorrhoids.

Other variations, embodiments and features of the present invention will become evident from the following detailed description, drawings and claims.

### BRIEF DESCRIPTION OF THE DRAWINGS

FIG. 1 illustrates an ingredient list for a natural gel/ointment according to the embodiments of the present invention;

FIG. 2 illustrates a flow chart detailing one treatment utilizing the natural gel/ointment according to the embodiments of the present invention;

FIG. 3 illustrates a cool-tip applicator for applying the gel/ointment according to the embodiments of the present invention; and

FIG. 4 illustrates a kit container designed to hold a container of the soaking agent along with the tablets and gel/ointment as described in the referenced co-pending applications.

### DETAILED DESCRIPTION

It will be appreciated by those of ordinary skill in the art that the invention can be embodied in other specific forms without departing from the spirit or essential character thereof. The presently disclosed embodiments are therefore considered in all respects to be illustrative and not restrictive.

A natural treatment for hemorrhoids has benefits for both the individual with the ailment and society as a whole. The benefit to society includes a decreased need for surgery, prescription drugs and other unnecessary unnatural treatments. The individual suffering from hemorrhoids benefits by being able to utilize an easy-to-apply, effective topical. Over-the-counter topical Preparation H® utilizes mineral oil (protectant), petrolatum (protectant), Phenylephrine (vasoconstrictor) and shark liver oil (protectant). Over-the-counter topical Tucks® utilizes a combination of mineral oil (protectant), Pramoxine HCL (pain relief) and Zinc Oxide (protectant). Over-the-counter topical Balneol® is essentially a moisturizer. Therefore, the active ingredients in most over-the-counter topical applications are utilized primarily to protect and treat pain in the area affected by hemorrhoids but do not effectively treat or prevent the hemorrhoids. Prescription drugs, like topical Analpram, utilize Pramoxine HCL (pain relief) and Hydrocortisone (steroidal anti-inflammatory), but still fail to treat hemorrhoids as well as the embodiments of the present invention. Another benefit to the over-the-counter natural gel/ointment is that is can be used in conjunction with the tablet described in co-pending patent application Ser. No. 12/______ and/or the soaking agent described in co-pending patent application Ser. No. 12/______. Any of the treatments alone or in combination facilitate the avoidance of expensive prescription drugs. In many instances today, patients are not able to afford prescription drugs. This is true for those without insurance or those having insurance given large deductibles and countless uncovered prescriptions.

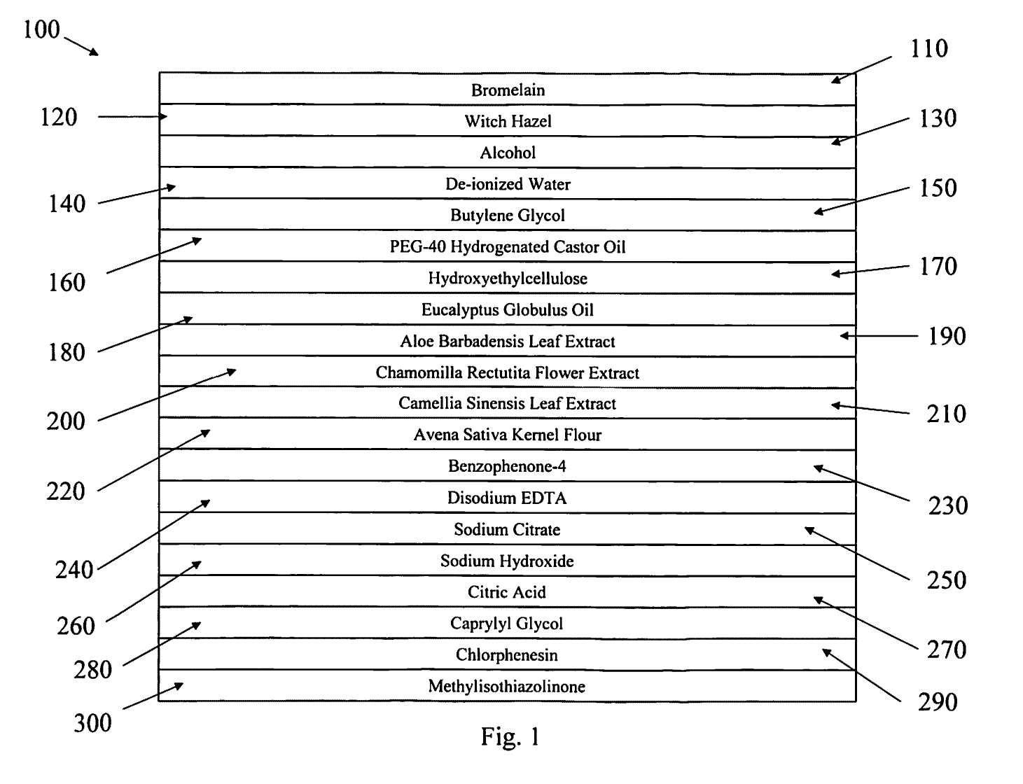

FIG. 1 depicts an ingredient list 100 for a first embodiment of the present invention. The two primary active ingredients comprise Bromelain 110 and Hamamelis Virginiana (commonly known as Witch Hazel) 120. The Bromelain 110 and Witch Hazel leaf 120 have an anti-inflammatory effect. When combined, the Bromelain 110 and Witch Hazel 120 have a synergistic effect when combined as evidenced by the case studies detailed below.

Bromelain is a mixture of protein-digesting (proteolytic) enzymes found in pineapples. Bromelain, which is derived from the stem and juice of the pineapple, was first isolated from the pineapple plant in the late 1800s. Witch Hazel is a low growing shrub native to North America. Witch Hazel has many uses including as an anti-inflammatory, astringent, toner and numbing agent.

In a first embodiment, as depicted in the ingredient list 100 a gel/ointment according to the embodiments of the present invention comprises secondary ingredients including alcohol 130, de-ionized water 140, Butylene Glycol 150, PEG-40 Hydrogenated Castor Oil 160, Hydroxyethylcellulose 170, Eucalyptus Globulus Oil 180, Aloe Barbadensis (Aloe Vera) Leaf Extract 190, Chamomilla Rectutita (Matricaria) Flower Extract 200, Camellia Sinensis (Oolong Tea) Leaf Extract 210, Avena Sativa (Oat) Kernel Flour 220, Benzophenone-4 230, Disodium EDTA 240, Sodium Citrate 250, Sodium Hydroxide 260, Citric Acid 270, Caprylyl Glycol 280, Chlorphenesin 290 and Methylisothiazolinone 300. Butylene Glycol 150 is a humectant and solubilzer. PEG-40 Hydrogenated Castor Oil 160 is an emollient. Hydroxyethylcellulose 170 is a gelling and thickening agent derived from cellulose. Eucalyptus Globulus Oil 180 is an essential oil. Aloe Barbadensis (Aloe Vera) Leaf Extract 190 is a soothing agent. Chamomilla Recutita (Matricaria) Flower Extract 200 acts as an antibacterial. Benzophenone-4 230 is a color agent. Disodium EDTA 240 is a chelating agent and a viscosity adjuster. Sodium Citrate 250 is an alkalinizing agent. Sodium Hydroxide 260 is an alkaline compound. Citric Acid 270 is a weak organic acid. Caprylyl Glycol 280 is a moisturizing or wetting agent and used for stabilization. Chlorphenesin 290 is an antifungal and antibacterial. Methylisothiazolinone 300 is a biocide and preservative. Those skilled in the art will recognize that other ingredients having the same properties may be used in place of, or in addition to, the aforementioned secondary ingredients. Also, certain of the listed secondary ingredients may be removed while maintaining the effectiveness of the gel/ointment. The ingredients are combined into a gel/ointment using conventional manufacturing and compounding techniques.

In one embodiment, the gel/ointment in accordance with the specifications listed above, are to be taken 3 times daily until hemorrhoid symptoms dissipate.

Table 1 details exemplary case studies. Advantageously, the results of utilizing the gel/ointment in accordance with the embodiments of the present invention have dramatic synergistic effects over usage of the active ingredients individually. In addition, a high majority of the patients expressed that the gel/ointment outperformed other hemorrhoid treatments.

TABLE 1

| | | | | | | | Tablet More | Tablet |
|---|---|---|---|---|---|---|---|---|
| | | | | Improvement | | OTC | Effective Than | Effectiveness |
| Age | Gender | Diagnosis | Symptoms | Time | | Products | OTC Meds? | 0-5 |
| 64 | Female | Hemorrhoids | Swelling & Pain | 7 | days | Analpram | Yes | 5 |
| 86 | Female | Hemorrhoids | Bleeding & Pain | 1 | day | N/A | N/A | 5 |
| 66 | Male | Hemorrhoids | Swelling | 1 | week | Prep H | Yes | 4 |
| N/A | Female | Hemorrhoids | Swelling | 1 | day | N/A | Yes | 5 |
| N/A | N/A | Hemorrhoids | Pain & Itching | 7 | days | Analpram | Yes | 3 |
| 42 | Female | Hemorrhoids | Pain | 2 | weeks | N/A | Yes | 3 |
| 80 | Male | Hemorrhoids | Swelling & Pain | 10 | days | Prep H | Yes | 5 |
| 78 | Male | Hemorrhoids | Bleeding & Pain | 2 | weeks | Analpram | Yes | 3 |

Table 1 lists the age and gender of a majority of patients. Also shown in Table 1 are the diagnosis, symptoms, time for results, over-the-counter medications used by the patients, each patient's opinion of whether the gel/ointment has better results than the over-the-counter medications and a rating score of effectiveness associated with the gel/ointment. Table 1 evidences that the gel/ointment treats the typical symptoms namely swelling, bleeding, pain and itching associated with hemorrhoids. Positive results from the gel/ointment were felt in a range of 1 day to 2 weeks. Each patient opined that the tablets treated hemorrhoids better than over-the-counter medications (e.g., Preparation H® (Prep H) and Analpram). In addition, the patients rated the treatment as an average of 4.125 out of 5 on the ratings scale. Such feedback, including the average rating, evidences a dramatic effect associated with the ingredients in the tablet form.

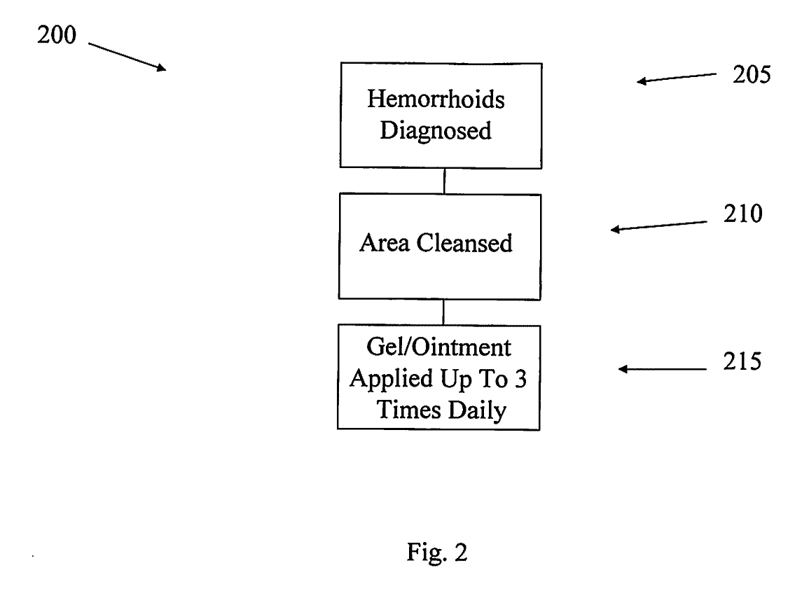

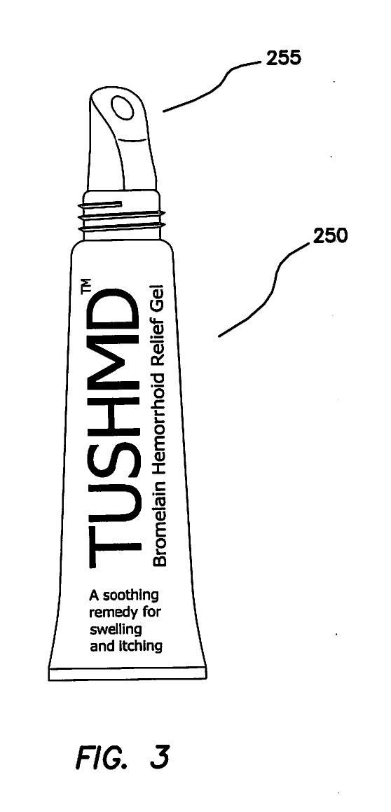

FIG. 2 shows a flow chart 200 detailing a method of treating hemorrhoids using the gel/ointment. At 205, hemorrhoids is diagnosed. At 210, the patient is instructed to cleanse the area. At 215, the gel/ointment is applied to the lower portion of the anal canal up to three times daily. In one embodiment, the gel/ointment is packaged in a squeezable tube 250 with a cool-touch application tip 255 as shown in FIG. 3. The cool-touch application tip 255 provides a sensitive, clean and sanitary means for applying the gel/ointment to the subject area.

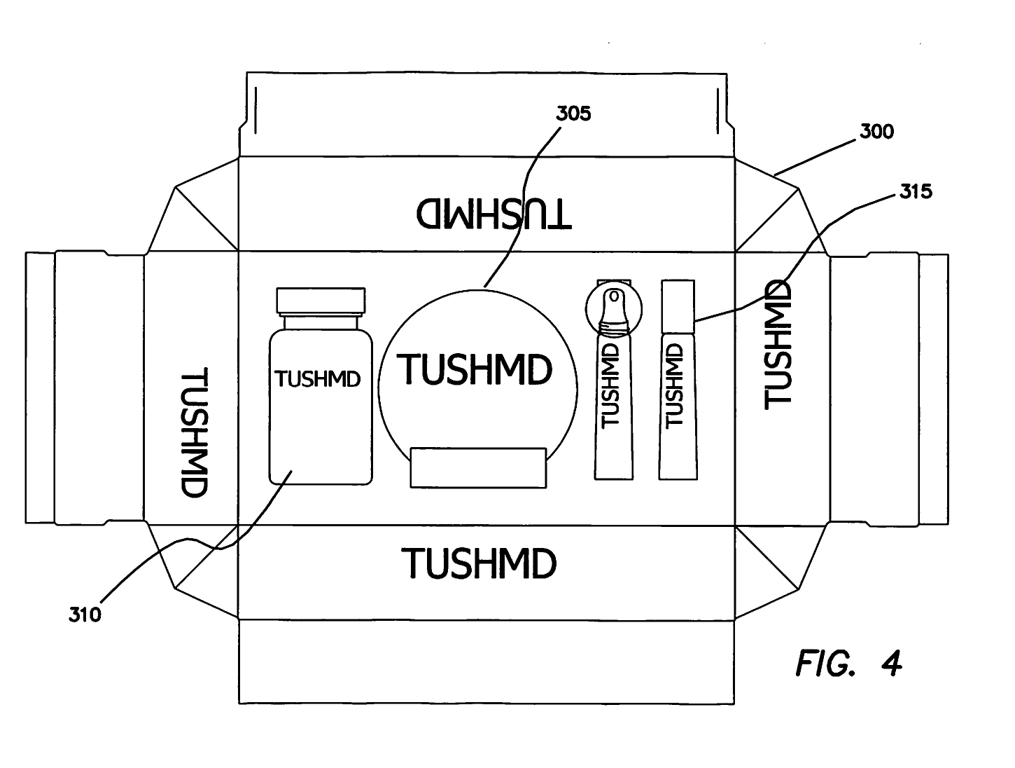

FIG. 4 shows a form 300 for constructing a box to hold a kit comprising individual containers for each of the soaking agent 305 (described in co-pending patent application Ser. No. 12/______), tablets 310 (described in co-pending patent application Ser. No. 12/______) and gel/ointment 315. Separately or alone, the soaking agent, tablets and gel/ointment effectively treat hemorrhoids. The treatment protocols for each delivery vehicle remain the same when each is used in combination as when used individually. In other words, all three treatment protocols can be used simultaneously without any negative side effects.

Although the invention has been described in detail with reference to several embodiments, additional variations and modifications exist within the scope and spirit of the invention as described and defined in the following claims.

I claim:

1. A topical medication for the treatment of hemorrhoids comprising:
Bromelain; and
one or more ingredients in combination with said Bromelain to form a gel or ointment.

2. The topical mediation of claim 1 wherein said one or more ingredients comprise one or more of the following: alcohol, de-ionized water, Butylene Glycol, PEG-40 Hydrogenated Castor Oil, Hydroxyethylcellulose, Eucalyptus Globulus Oil, Aloe Barbadensis Leaf Extract, Chamomilla Rectutita Flower Extract, Camellia Sinensis Leaf Extract, Avena Sativa Kernel Flour, Benzophenone-4, Disodium EDTA, Sodium Citrate, Sodium Hydroxide, Citric Acid, Caprylyl Glycol, Chlorphenesin and Methylisothiazolinone.

3. A topical medication for the treatment of hemorrhoids comprising:
Bromelain; and
Hamamelis Virginiana.

4. The topical mediation of claim 3 further comprising one or more of the following ingredients: alcohol, de-ionized water, Butylene Glycol, PEG-40 Hydrogenated Castor Oil, Hydroxyethylcellulose, Eucalyptus Globulus Oil, Aloe Barbadensis Leaf Extract, Chamomilla Rectutita Flower Extract, Camellia Sinensis Leaf Extract, Avena Sativa Kernel Flour, Benzophenone-4, Disodium EDTA, Sodium Citrate, Sodium Hydroxide, Citric Acid, Caprylyl Glycol, Chlorphenesin and Methylisothiazolinone.
